(19) United States
(12) Patent Application Publication
(10) Pub. No.: US 2015/0133464 A1
(43) Pub. Date: May 14, 2015

(54) Injectable particle

(71) Applicant: David Wong, Milpitas, CA (US)

(72) Inventor: David Wong, Milpitas, CA (US)

(73) Assignee: David Wong, Milpitas, CA (US)

(21) Appl. No.: 14/611,268

(22) Filed: Feb. 1, 2015

(51) Int. Cl. A61K 9/14; A61K 9/00; A61K 31/506; A61K 31/44

(52) U.S. Cl. CPC A61K 9/146; A61K 31/44; A61K 9/0019; A61K 31/506; A61K 9/145; A61K 9/143
USPC 514/252.19; 514/350

(57) ABSTRACT

The present invention generally relates to an novel injectable particle composition essentially consisting of: (a) a population of domains, wherein each domain essentially consists of a drug matrix and a population of subdomains, wherein the subdomains are evenly distributed in the drug matrix, wherein each subdomain essentially consists of an ionizable excipient, and (b) a rate controlling matrix comprising a biocompatible polymer and optionally an excipient. The particle is for the use of cancer therapy.

### TECHNICAL FIELD

The present invention generally relates to an novel injectable particle composition essentially consisting of: (a) a population of domains, wherein each domain essentially consists of a drug matrix and a population of subdomains, wherein the subdomains are evenly distributed in the drug matrix, wherein each subdomain essentially consists of an ionizable excipient, and (b) a rate controlling matrix comprising a biocompatible polymer and optionally an excipient. The particle is for the use of cancer therapy.

### BACKGROUND OF THE INVENTION

Ionizable excipients such as amino acids and water-soluble carbonates, are popularly found in polymeric matrix systems for sustained release of drugs. Leaching out of such ionizable excipients leads to a porous structure of the particle matrix, consequently, facilitating the drug release. However, early leaching of a significant amount of ionizable excipients before reaching the action site is undesirable, as this will lead to a significant drug release before reaching the action site. Thus, there is a need of alternative polymeric matrix systems for sustained release of drugs.

### BRIEF SUMMARY OF THE INVENTION

The inventor has discovered an novel injectable particle composition consisting of: (a) a population of domains, wherein each domain essentially consists of a drug matrix and a population of subdomains, wherein each subdomain essentially consists of an ionizable excipient, and (b) a rate controlling matrix comprising a biocompatible polymer and optionally an excipient. The particle is used as an injectable dosage form for cancer treatment.

Accordingly, the present invention relates to an novel injectable particle composition essentially consisting of: (a) a population of domains, wherein each domain essentially consists of a drug matrix and a population of subdomains, wherein the subdomains are evenly distributed in the drug matrix, wherein each subdomain essentially consists of an ionizable excipient, and (b) a rate controlling matrix comprising a biocompatible polymer and optionally an excipient. In one aspect, the particle is prepared by a method consisting of 2-3 cycles of “dispersion, precipitation, and solvent evaporation”, and then spray-drying.

### BRIEF DESCRIPTION OF THE FIGURE

FIG. 1. An illustration of the injectable particle. The injectable particle contains a population of domains and a rate controlling matrix, wherein each domain contains a population of subdomains and a drug matrix; wherein the subdomains are distributed in the drug matrix.

### DETAILED DESCRIPTION OF THE INVENTION

### Definitions

As used herein, the term “drug” means a compound intended for use in treatment of cancer, in man or other animals. The desired properties of the preferred drugs are: (1) being water-insoluble or sparingly soluble in water, (2) being soluble in acetone or a mixture of acetone and 2-butanol, and (3) possessing anti-cancer activities. Examples of the preferred drugs include, but are not limited to, sorafenib tosylate and dasatinib.

Singular forms included in the claims such as “a”, “an” and “the” include the plural reference unless expressly stated or the context clearly indicates otherwise.

“Optional” or “optionally” means that the subsequently described circumstance may or may not occur, so that the description includes instances where the circumstance occurs and instances where it does not.

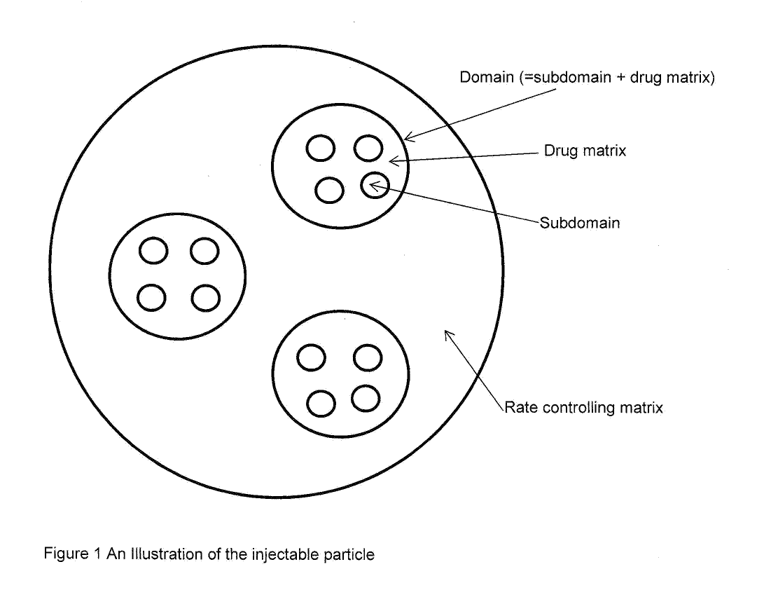

Domains are “spots” distributed inside the particle. From another point of view, they are dispersed evenly in the rate controlling matrix. (See FIG. 1) Domains are formed by a precipitation process of the drug substance in the polymeric solution. Each domain essentially consists of a drug matrix and a population of subdomains.

The term, drug matrix, means a matrix consisting of one or more drug(s), wherein subdomains are distributed in this matrix. (See FIG. 1) Thus, “a drug matrix” may mean: (1) a matrix essentially consisting of sorafenib tosylate, and subdomains are distributed in this matrix, (2) a matrix essentially consisting of dasatnib, and subdomains are distributed in this matrix, or (3) a matrix essentially consisting of sorafenib tosylate and dasatinib, and subdomains are distributed in this matrix. In one aspect, if the drug is specified in the matrix description, such as “sorafenib tosylate matrix”, then, that matrix consists of sorafenib tosylate, and subdomains are distributed in this matrix. A drug matrix does not contain an excipient, but, it may contain a trace amount of the ionizable excipient involved in the previous steps of the manufacturing process.

Subdomains are “spots” distributed in the domain. They are formed when an ionizable excipient precipitates out in the drug solution. Thus, the subdomains are surrounded by the drug matrix, this arrangement of subdomains and the drug matrix constitutes the domain. (FIG. 1) In this invention, subdomains are also described as they are distributed in the drug matrix. The selected ionizable excipients are ethylenediaminetetraacetic acid (EDTA), metal carbonates and amino acids.

The term, rate controlling matrix, refers to the matrix of the injectable particle, which allows the sustained release fashion of a drug. The rate controlling matrix is composed of a biodegradable polymer. Domains are dispersed in the rate controlling matrix. While, under certain circumstances, a trace amount of the drug may also be dispersed “molecularly” in the rate controlling matrix.

The term, injectable particles, refers to solid particles with an average size of about 5 nm to about 50,000 nm, and these solid particles are intended to be delivered via intravenous injection, hepatic arterial infusion or other means for chemotherapy.

The term, metal chloride, refers to a compound releases a metal ion and chloride ions after dissolving in an aqueous medium. The preferred metal chlorides in this invention are divalent and trivalent metal chlorides, such as copper and iron chlorides. The most preferred metal chlorides are calcium chloride and iron (III) chloride (or ferric chloride).

### The Invention

The key ingredients of the invention are sorafenib tosylate, dazatinib, an ionizable excipient, and a biocompatible polymer.

Sorafenib tosylate and dasatinib are kinase inhibitors; they are useful on treating various cancer-diseases. This invention can also be applied to other drugs with a similar solubility profile for other treatments and/or oral dosage forms.

Ionizable excipients are drug release modulators. The desired properties of ionizable excipients are (1) carrying charges in aqueous media and (2) being not soluble in acetone. Selected ionizable excipients are water-soluble carbonates, ethylenediaminetetraacetic acid (EDTA), and amino acids with properties similar to isoleucine. The most preferred ionizable excipient further carries one or both of the following characters: (1) being a gas forming agent, or (2) potentially being able to lower the side effects of the drug substance. The most preferred ionizable excipients are isoleucine and sodium carbonate.

Hyaluronic acid (HA) is a polysaccharide composed of alternating molecules of N-acetyl glucosamine and D-glucuronic acid. It can be found within collagen throughout the body. HA sodium salt from Streptococcus equi is available at SIGMA-ALDRICH®. The preferred molecular weight of HA is slightly below 3,500 Da.

Chen G et al, Bioconjug Chem 1997 September-October, 8(5) 730-4 suggests that HA can be sulfated by a sulfur trioxide-pyridine complex to form sulfated HA. Barbucci R. et al, J. Thromb Thrombolysis, 1998 September; 6(2): 109-115, suggest sulfation of the —OH groups of HA to prepare different sHA derivatives.

U.S. Pat. No. 6,673,919 B2 describes a detailed method for the preparation of the acetylated HA (aHA). While, Carmela Saturnino et al, BioMed Research International; Volume 2014 (2014), also suggest a method for the preparation of aHA: (1) adding catalytic amount of 4-dimethylaminopyridine (DMAP) and an excess of acetic anhydride to a stirred cold solution of 500 mg sodium hyaluronate in 10 ml of toluene. (2) stirring the mixture at reflux, under nitrogen, for 24 hours and then concentrate the mixture under reduced pressure, (3) purifying the solid residue by silica gel chromatography using dichloromethane and methanol (in a ratio of 9:1) as eluent, and then obtaining the pure compound as white solid.

Poly(lactide-co-glycolide) (PLGA) is a biocompatible polymer. PLGA in ratios of 50:50, 65:35; 75:25 and 85:15 (lactide:glycolide) with different molecular weights, are available at SIGMA-ALDRICH®. Parameters such as polymer molecular weight, ratio of lactide to glycolide, drug properties and drug concentration determine the dosage and the release rate.

Huang, J et al, Biomaterials 35 (2014) 550-566), describes a detailed method for preparing PLGA-HA copolymer. Basically, the synthesis is via an end to end coupling technology: synthesis of amino-functional HA, synthesis of N-Hydroxysuccinimide PLGA (PLGA-NHS), and finally synthesis of PLGA-HA. Similar process can be applied to prepare PLGA-aHA and PLGA-sHA.

The present invention provides an novel injectable particle composition essentially consisting of: (a) a population of domains, wherein each domain essentially consists of a drug matrix and a population of subdomains, wherein the subdomains are evenly distributed in the drug matrix, wherein each subdomain essentially consists of an ionizable excipient, and (b) a rate controlling matrix comprising a biocompatible polymer and optionally an excipient. Consequently, the particle may have one or more of the following characteristics: (1) a population of domains dispersed inside a particle, (2) a population of subdomains dispersed inside a domain, (3) the particle size is about 5 nm to about 50,000 nm, and (4) the particle is for the use of cancer therapy.

The injectable particles are prepared by the following steps: (1) dissolving an ionizable excipient in water, (2) dissolving a drug in an acetone, (3) dispersing the solution formed of Step 1 in the solution formed of Step 2, (4) mixing the dispersion of Step 3 for a few hours to precipitate out the ionizable excipient, (5) dissolving PLGA (or PLGA-HA, or PLGA-sHA or PLGA-aHA) in a mixture of acetone and water to form MIXTURE 1, (6) dispersing the suspension formed of Step 4 in the MIXTURE 1, (7) mixing the mixture of Step 6 well, for a few hours, (8) dispersing the mixture of Step 7 into an aqueous medium, (9) mixing well for a few hours, and then (10) spray-drying the dispersion of Step 9. Alternatively, the mixture of Step 4 is dispersed in an aHA solution (optionally with a metal chloride and/or polyvinylpyrrolidone), then spray-dried to form particles. Dialysis may be used to facilitate the solvent removal for the precipitation of the above process, if applicable. Otherwise, evaporation of solvent is the standard method for removing solvent. The standard preparation method of the invention involves dispersion, precipitation and solvent evaporation, which has been fully described in literatures. Nagavarma BV N et al, Asian Journal of Pharmaceutical and Clinical Research 2012, 5(3): 16-23, describe nanoprecipitation and solvent displacement mehod for preparing nanoparticles of water-soluble drugs in a water-miscible solvent. While, Do Hyung Kim et al, Nanoscale Research Letters 2012, 7:91, describes a preparation method of sorafenib nanoparticles by nanoprecipitation-dialysis. Further, Manisha Khemani et al, Annals of Biological Research, 2012, 3(9): 4414-4419, describes a method for preparing nanoparticles comprising doxorubicin HCl and PLGA. Kim et al, Int J Nanomedicine 2011, 6: 2621-2631 teaches a method for preparing nanoparticles comprising celecoxib and PLGA. Finally, U.S. Pat. No. 8,303,992 and U.S. Pat. No. 5,674,531 teach a method of spray-drying dispersions to form particles, and U.S. Pat. No. 7,901,711 teaches a method of freeze-drying of a mixture to form particles. However, alternative nanoparticle preparation methods, such as electrospray, critical fluid etc., can be used as substitutes for the method described above.

Accordingly, the present invention provides an injectable particle essentially consisting of: (a) a population of domains, wherein each domain essentially consists of a drug matrix and a population of subdomains, wherein the subdomains are evenly distributed in the drug matrix, wherein the drug matrix essentially consists of a drug, and wherein the drug is selected from a group consisting of sorafenib tosylate and dasatinib, wherein each subdomain essentially consists of an ionizable excipient, and wherein the ionizable excipient is selected from the group consisting of isoleucine and sodium carbonate, and (b) a rate controlling matrix comprising a biocompatible polymer and optionally an excipient, wherein the biocompatible polymer is selected from a group consisting of acetylated hyaluronic acid and poly(lactide-co-glycolide) copolymer. In one aspect, the biocompatible polymer is poly(lactide-co-glycolide) copolymer. The poly(lactide-co-glycolide) copolymer may be conjugated with a hyaluronic acid. Before the conjugation process, hyaluronic acid may first be acetylated or sulfated. In another aspect, the biocompatible polymer is acetylated hyaluronic acid. In this aspect, the rate controlling matrix may further comprise a metal chloride, and this metal chloride is selected from the group consisting of calcium chloride and iron chloride. And, examples of excipients include, but are not limited to, butylated hydroxyanisole, butylated hydroxytoluene, calcium carbonate, calcium phosphate, calcium sulfate, carboxymethylcellulose, crospovidone, cyclodextrin, dextrin, dextrose, glucose, gelatin, polyethylene glycol, lactose, mannitol, mineral oil, polysorbate, polyvinyl alcohol, polyvinylpyrrolidone, silicone, sodium acetate, sodium benzoate, sodium benzoate, sodium citrate, sodium lauryl sulfate, sodium thioglycolate, sorbitol, sucrose, trisodium citrate, and mixtures thereof. The domains are evenly distributed in the rate controlling matrix.

In one embodiment, the present invention provides a novel injectable particle essentially consisting of: (a) a population of domains, wherein each domain essentially consists of a sorafenib tosylate matrix and a population of subdomains, wherein the subdomains are evenly distributed in the sorafenib tosylate matrix, wherein each subdomain essentially consists of isoleucine, and (b) a rate controlling matrix essentially consisting of acetylated hyaluronic acid, optionally iron chloride, and optionally polyvinylpyrrolidone, and wherein the domains are dispersed in the rate controlling matrix.

In another embodiment, the present invention provides a novel injectable particle essentially consisting of: (a) a population of domains, wherein each domain essentially consists of a sorafenib tosylate matrix and a population of subdomains, wherein the subdomains are distributed evenly in the sorafenib tosylate matrix, wherein each subdomain essentially consists of sodium carbonate, and (b) a rate controlling matrix essentially consisting of acetylated hyaluronic acid, optionally iron chloride, and optionally polyvinylpyrrolidone, and wherein the domains are dispersed in the rate controlling matrix.

In another embodiment, the present invention provides a novel injectable particle essentially consisting of: (a) a population of domains, wherein each domain essentially consists of a drug matrix and a population of subdomains, wherein the drug matrix essentially consists of a mixture of sorafenib tosylate and dasatinib, wherein the subdomains are distributed evenly in the drug matrix, and wherein each subdomain essentially consists of sodium carbonate, and (b) a rate controlling matrix essentially consisting of acetylated hyaluronic acid, optionally iron chloride, and optionally polyvinylpyrrolidone, and wherein the domains are dispersed in the rate controlling matrix.

It is preferably that a small portion of the drug substance is molecularly dispersed in the rate controlling matrix in all the embodiments discussed above.

### EXAMPLES OF INVENTION

The foregoing examples are illustrative embodiments of the invention and are merely exemplary. A person skilled in the art may make variations and modifications without deviating from the spirit and scope of the invention. All such modifications and variations are intended to be included within the scope of the invention.

### Example 1

Isoleucine is dissolved in water to form isoleucine solution (aq.). Soratenib tosylate is dissolved in acetone to form sorafenib tosylate solution. Isoleucine solution (aq.), 1 ml, is dispersed in 30 ml of sorafenib tosylate solution. The mixture is mixed vigorously for a few hours to form MIXTURE 1. PLGA is dissolved in a mixture of acetone and ethanol to form PLGA solution. 1 ml of MIXTURE 1 is dispersed in 100 ml of the PLGA solution. The mixture is mixed vigorously for a few hours to form MIXTURE 2. 50 mL of MIXTURE 2 is dispersed in 1 L of deionized water. The mixture is mixed vigorously for a few hours for solvent evaporation. The mixture is then spray-dried to form particles.

### Example 2

EDTA is dissolved in a phosphate buffer to form an isoleucine solution (aq.). Soratenib tosylate is dissolved in acetone to form sorafenib tosylate solution. EDTA solution (aq.), 1 ml, is dispersed in 30 ml of sorafenib tosylate solution. The mixture is mixed vigorously for a few hours to form MIXTURE 1. PLGA-HA is dissolved in a mixture of acetone and ethanol to form PLGA-HA solution. 1 ml of MIXTURE 1 is dispersed in 100 ml of the PLGA-HA solution. The mixture is mixed vigorously for a few hours to form MIXTURE 2. 50 mL of MIXTURE 2 is dispersed in 1 L of deionized water. The mixture is mixed vigorously for a few hours for solvent evaporation. The mixture is then spray-dried to form particles.

### Example 3

Sodium carbonate is dissolved in water to form a sodium carbonate solution (aq.). Soratenib tosylate is dissolved in acetone to form sorafenib tosylate solution. Sodium carbonate solution (aq.), 1 ml, is dispersed in 30 ml of sorafenib tosylate solution. The mixture is mixed vigorously for a few hours to form MIXTURE 1. PLGA-HA is dissolved in a mixture of acetone and ethanol to form PLGA-HA solution. 1 ml of MIXTURE 1 is dispersed in 100 ml of the PLGA-HA solution. The mixture is mixed vigorously for a few hours to form MIXTURE 2. 50 mL of MIXTURE 2 is dispersed in 1 L of a polyethylene glycol solution (0.3%). The mixture is mixed vigorously for a few hours for solvent evaporation. The mixture is then spray-dried to form particles.

### Example 4

Sodium carbonate is dissolved in water to form a sodium carbonate solution (aq.). Soratenib tosylate is dissolved in acetone to form sorafenib tosylate solution. Sodium carbonate solution (aq.), 1 ml, is dispersed in 30 ml of sorafenib tosylate solution. The mixture is mixed vigorously for a few hours to form MIXTURE 1. PLGA is dissolved in a mixture of acetone and ethanol to form PLGA-HA solution. 1 ml of MIXTURE 1 is dispersed in 100 ml of the PLGA-HA solution. The mixture is mixed vigorously for a few hours to form MIXTURE 2. 50 mL of MIXTURE 2 is dispersed in 1 L of a mixture of polyethylene glycol solution (0.3%) and polysorbate (0.1%). The mixture is mixed vigorously for a few hours for solvent evaporation. The mixture is then spray-dried to form particles.

### Example 5

Sodium carbonate is dissolved in water to form a sodium carbonate solution (aq.). Soratenib tosylate is dissolved in acetone to form sorafenib tosylate solution. Sodium carbonate solution (aq.), 1 ml, is dispersed in 30 ml of sorafenib tosylate solution. The mixture is mixed vigorously for a few hours to form MIXTURE 1. aHA and ferric chloride are dissolved in an aqueous medium to form MIXTURE 2. MIXTURE 1 is dispersed in 1000 ml of MIXTURE 2. The mixture is mixed vigorously for a few hours. The mixture is then spray-dried to form particles.

### Example 6

Isoleucine is dissolved in water to form an isoleucine solution (aq.). Soratenib tosylate is dissolved in acetone to form sorafenib tosylate solution. Isoleucine solution (aq.), 1 ml, is dispersed in 30 ml of sorafenib tosylate solution. The mixture is mixed vigorously for a few hours to form MIXTURE 1. aHA and polyvinylpyrrolidone (PVP) are dissolved in an aqueous medium to form aHA/PVP solution. MIXTURE 1 is dispersed in 1000 ml of the aHA/PVP solution. The mixture is mixed vigorously for a few hours. The mixture is then spray-dried to form particles.

### Example 7

Sodium carbonate is dissolved in water to form a sodium carbonate solution (aq.). Soratenib tosylate is dissolved in acetone to form sorafenib tosylate solution. Sodium carbonate solution (aq.), 1 ml, is dispersed in 30 ml of sorafenib tosylate solution. The mixture is mixed vigorously for a few hours to form MIXTURE 1. PLGA-aHA is dissolved in a mixture of acetone and ethanol to form PLGA-aHA solution. 1 ml of MIXTURE 1 is dispersed in 100 ml of the PLGA-aHA solution. The mixture is mixed vigorously for a few hours to form MIXTURE 2. 50 mL of MIXTURE 2 is dispersed in 1 L of a polyethylene glycol solution (0.3%). The mixture is mixed vigorously for a few hours for solvent evaporation. The mixture is then spray-dried to form the particles.

### Example 8

Sodium carbonate is dissolved in water to form a sodium carbonate solution (aq.). Soratenib tosylate is dissolved in acetone to form sorafenib tosylate solution. Sodium carbonate solution (aq.), 1 ml, is dispersed in 30 ml of sorafenib tosylate solution. The mixture is mixed vigorously for a few hours to form MIXTURE 1. PLGA-sHA is dissolved in a mixture of acetone and ethanol to form PLGA-sHA solution. 1 ml of MIXTURE 1 is dispersed in 100 ml of the PLGA-sHA solution. The mixture is mixed vigorously for a few hours to form MIXTURE 2. 50 mL of MIXTURE 2 is dispersed in 1 L of a polyethylene glycol solution (0.3%). The mixture is mixed vigorously for a few hours for solvent evaporation. The mixture is then spray-dried to form the particles.

### Example 9

Isoleucine is dissolved in water to form an isoleucine solution (aq.). Dasatinib is dissolved in a mixture of acetone and 2-butanol to form dasatinib solution. Isoleucine solution (aq.), 1 ml, is dispersed in 30 ml of dasatinib solution. The mixture is mixed vigorously for a few hours to form MIXTURE 1. aHA and calcium chloride are dissolved in an aqueous medium to form aHA/calcium chloride solution. MIXTURE 1 is dispersed in 1000 ml of the aHA/calcium chloride solution. The mixture is mixed vigorously for a few hours. The mixture is then spray-dried to form particles.

### Example 10

Sodium carbonate is dissolved in water to form a sodium carbonate solution (aq.). Dasatinib is dissolved in a mixture of acetone and 2-butanol to form dasatinib solution. Sodium carbonate solution (aq.), 1 ml, is dispersed in 30 ml of dasatinib solution. The mixture is mixed vigorously for a few hours to form MIXTURE 1. aHA and calcium chloride are dissolved in an aqueous medium to form aHA/calcium chloride solution. MIXTURE 1 is dispersed in 1000 ml of the aHA/calcium chloride solution. The mixture is mixed vigorously for a few hours. The mixture is than spray-dried to form particles.

### Example 11

Sodium carbonate is dissolved in water to form a sodium carbonate solution (aq.). Dasatinib and sorafenib tosylate are co-dissolved in a mixture of acetone and 2-butanol to form a drug solution. Sodium carbonate solution (aq.), 1 ml, is dispersed in 30 ml of the drug solution. The mixture is mixed vigorously for a few hours to form MIXTURE 1. aHA and iron chloride are dissolved in an aqueous medium to form an aHA/iron chloride solution. MIXTURE 1 is dispersed in 1000 ml of the aHA/iron chloride solution. The mixture is mixed vigorously for a few hours. The mixture is then spray-dried to form particles.

I claim:

1. An injectable particle essentially consisting of: (a) a population of domains, wherein each domain essentially consists of a drug matrix and a population of subdomains, wherein the subdomains are evenly distributed in the drug matrix, wherein the drug matrix essentially consists of a drug, and wherein the drug is selected from a group consisting of sorafenib tosylate and dasatinib, wherein each subdomain essentially consists of an ionizable excipient, and wherein the ionizable excipient is selected from the group consisting of isoleucine and sodium carbonate, and (b) a rate controlling matrix comprising a biocompatible polymer and optionally an excipient, wherein the biocompatible polymer is selected from a group consisting of acetylated hyaluronic acid and poly(lactide-co-glycolide) copolymer, and wherein the domains are evenly distributed in the rate controlling matrix.

2. The injectable particle according to claim 1, wherein the biocompatible polymer is poly(lactide-co-glycolide) copolymer.

3. The injectable particle according to claim 1, wherein the biocompatible polymer is acetylated hyaluronic acid, wherein the rate controlling matrix may further comprise a metal chloride, and wherein the metal chloride is selected from the group consisting of calcium chloride and iron chloride.

4. The injectable particle according to claim 2, wherein the poly(lactide-co-glycolide) copolymer is conjugated with a hyaluronic acid.

5. The injectable particle composition according to claim 4, wherein the hyaluronic acid is acetylated.

6. The injectable particle composition according to claim 4, wherein the hyaluronic acid is sulfated.

7. An injectable particle essentially consisting of: (a) a population of domains, wherein each domain essentially consists of a sorafenib tosylate matrix and a population of subdomains, wherein the subdomains are evenly distributed in the sorafenib tosylate matrix, and wherein each subdomain essentially consists of isoleucine, and (b) a rate controlling matrix essentially consisting of acetylated hyaluronic acid, optionally iron chloride, and optionally polyvinylpyrrolidone, and wherein the domains are dispersed in the rate controlling matrix.

8. An injectable particle essentially consisting of: (a) a population of domains, wherein each domain essentially consists of a drug matrix and a population of subdomains, wherein the drug matrix essentially consists of a mixture of sorafenib tosylate and dasatinib, wherein the subdomains are distributed evenly in the drug matrix, and wherein each subdomain essentially consists of sodium carbonate, and (b) a rate controlling matrix essentially consisting of acetylated hyaluronic acid, optionally iron chloride, and optionally polyvinylpyrrolidone, and wherein the domains are dispersed in the rate controlling matrix.
